(19) United States
(12) Patent Application Publication
(10) Pub. No.: US 2015/0362498 A1
(43) Pub. Date: Dec. 17, 2015

(54) METHOD OF DETECTING C-MET GENE AMPLIFICATION CELL

(71) Applicant: FUJIREBIO INC., Tokyo (JP)

(72) Inventors: Nobuyuki ISE, Tokyo (JP); Kazuya OMI, Tokyo (JP); Shigeki HIGASHIYAMA, Ehime (JP); Nobuaki TAKEMORI, Ehime (JP)

(73) Assignee: FUJIREBIO INC., Shinjuku-ku Tokyo (JP)

(21) Appl. No.: 14/761,457

(22) PCT Filed: Jan. 21, 2014
(86) PCT No.: PCT/JP2014/051088

§ 371 Date: Jul. 16, 2015

(30) Foreign Application Priority Data
Feb. 15, 2013 (JP) 2013-027404

(51) Int. Cl. G01N 33/574; C07K 16/32

(52) U.S. Cl. CPC G01N 33/57492; C07K 16/32; G01N 2333/71

(57) ABSTRACT

The present invention provides a method for detecting a c-MET gene amplification cell. Specifically, the present invention provides a method for detecting a c-MET gene amplification cell, comprising measuring a soluble c-MET in a sample utilizing a region specific for the soluble c-MET, wherein the soluble c-MET is formed by cleavage between the lysine residue (K) and the valine residue (V) in the partial region GKVI (SEQ ID NO:2) in a c-MET extracellular domain.

### TECHNICAL FIELD

The present invention relates to a method for detecting a c-MET gene amplification cell, and the like.

### BACKGROUND ART

It is shown that growth of a certain cancer can be inhibited by inhibiting a receptor-type tyrosine kinase c-MET that is one of oncogenes, and development of a c-MET inhibitor as an anticancer agent has been advanced. Although there are many unclear points so far about the cancers for which an inhibitor against the c-MET is effective, the cancers associated with abnormal activation of a c-MET (e.g., amplification of the c-MET gene, and mutation of the c-MET gene) are focused as promising candidates. Therefore, it is conceivable that diagnosis of nature of cancer attributes enables selection of the cancers to be treated with the c-MET inhibitor.

For cancers associated with amplification of the c-MET gene among the cancers associated with the abnormal activation of c-MET, a FISH method which can directly detecting the gene amplification is promising, but the FISH method is problematic for automation since it requires cumbersome steps in treatments of cancer cells and detection of the gene amplification. Thus, for the cancers associated with the amplification of the c-MET gene, it is expected to develop a more convenient diagnosis method.

As a phenomenon associated with the c-MET gene amplification, a phenomenon in which c-MET is cleaved in its extracellular domain, thereby soluble c-MET is extracellularly secreted and further migrates in blood was found (Patent Literature 1). However, the presence of a soluble splicing variant consisting of a c-MET extracellular domain-constituting polypeptide alone (Non-patent Literature 1) and a phenomenon in which c-MET is cleaved with a different protease in its extracellular domain (Non-patent Literatures 2 and 3) were reported. Thus, it is thought that cells not associated with the c-MET gene amplification also secret the soluble splicing variant and/or different soluble c-MET into blood. Therefore, in order to diagnose the cancer associated with the c-MET gene amplification with a high degree of accuracy, it is thought that it is necessary to distinctively measure a specific soluble c-MET that is to be secreted from cancer cells associated with the c-MET gene amplification from a different soluble c-MET and a soluble splicing variant that are to be secreted from cancer cells not associated with the c-MET gene amplification.

### PRIOR ART LITERATURE

### Patent Literature

Patent Literature 1: International Publication WO2011/125458

### Non-Patent Literature

Non-patent Literature 1: Jin P. et al., Arthritis Res. Ther., 2008; 10(4): R73 (Available online http://arthritis-research.com/content/10/4/R73)
Non-patent Literature 2: Kopitz C. et al., Cancer Res., 2007; 67: 8615-8623
Non-patent Literature 3: Schelter F. et al., J Biol. Chem., 2010; 285(34): 26335-40

### DISCLOSURE OF INVENTION

### Problem to be Solved by the Invention

It is an object of the present invention to provide a method for detecting a c-MET gene amplification cell.

### Means for Solving Problem

As a result of an extensive study, the present inventors have found that a specific soluble c-MET fragment specifically secreted by a c-MET gene amplification cell is formed by cleavage between the lysine residue (K) and the valine residue (V) in the partial region GKVI (SEQ ID NO:2) in a c-MET extracellular domain, this cleavage is predominantly caused by ADAM17, the c-MET gene amplification cell can be specifically detected by specifically measuring the specific soluble c-MET fragment in a sample, and the like, and completed the present invention.

Accordingly, the present invention is as follows:

[1] A method for detecting a c-MET gene amplification cell, comprising measuring a soluble c-MET in a sample utilizing a region specific for the soluble c-MET, wherein the soluble c-MET is formed by cleavage between the lysine residue (K) and the valine residue (V) in the partial region GKVI (SEQ ID NO:2) in a c-MET extracellular domain.
[2] The method of [1], which is a method of examining a cancer.
[3] The method of [1] or [2], wherein the sample is derived from a human.
[4] The method of any of [1] to [3], wherein the sample is a liquid sample.
[5] The method of any of [1] to [4], wherein the c-MET gene amplification cell is a lung cancer cell.
[6] The method of any of [1] to [5], comprising:
(1) treating the sample with a protease; and
(2) measuring a fragment of the soluble c-MET which has the dipeptide unit GK at its C-terminus.
[7] The method of any of [1] to [5], wherein the measurement of the soluble c-MET is carried out by detecting the fragment of the soluble c-MET which has the dipeptide unit GK at its C-terminus by mass spectrometry.
[8] The method of [6] or [7], wherein the soluble c-MET fragment is a peptide consisting of an amino acid sequence of QAISSTVLGK (SEQ ID NO:10) or KQAISSTVLGK (SEQ ID NO:11).
[9] An affinity substance for a region specific for a soluble c-MET formed by cleavage between the lysine residue (K) and the valine residue (V) in the partial region GKVI (SEQ ID NO:2) in a c-MET extracellular domain.
[10] The affinity substance of [9], wherein the affinity substance is an antibody.
[11] A diagnostic reagent for cancers, comprising an affinity substance for a region specific for a soluble c-MET formed by cleavage between the lysine residue (K) and the valine residue (V) in the partial region GKVI (SEQ ID NO:2) in a c-MET extracellular domain.

### Effect of the Invention

The method of the present invention is useful for examining the cancers associated with the c-MET gene amplification since it can specifically detect the c-MET gene amplification cell. The method of the present invention can also conveniently determine a therapeutic effect of an anticancer agent (e.g., molecular-targeted drugs such as a c-MET inhibitor and an EGFR inhibitor) on cancers, and can conveniently monitor the therapeutic effect of the anticancer agent on the cancers.

### BRIEF DESCRIPTION OF DRAWINGS

FIG. 1 is a view showing inhibition of c-MET secretion by knockdown of an ADAM17 gene. si-N.C.: negative control si-RNA; si-AD10: si-RNA targeting an ADAM10 gene; siAD17: si-RNA targeting the ADAM17 gene;

FIG. 2 is view showing detection of a c-MET extracellular domain fragment in culture supernatant; and

FIG. 3 is a view showing detection of a peptide fragment having 922K at its C-terminus, which is formed by digestion with trypsin, Lys-C and chymotrypsin.

### EMBODIMENT FOR CARRYING OUT THE INVENTION

The present invention provides a method for detecting a c-MET gene amplification cell.

The method of the present invention comprises measuring a soluble c-MET in a sample utilizing a region specific for the soluble c-MET, wherein the soluble c-MET is formed by cleavage between the lysine residue (K) and the valine residue (V) in the partial region GKVI (SEQ ID NO:2) in a c-MET extracellular domain.

c-MET is a receptor of a hepatocyte growth factor (HGF) and activates an intracellular signaling by binding with the HGF. c-MET is composed of the α chain and β chain generated by cleavage of a precursor of c-MET (pro c-MET) and forms a dimer by a disulfide linkage. c-MET is a receptor penetrating a cell membrane and the entire α chain and a part of the β chain are present extracellularly (see e.g., Mark et al., The Journal of Biological Chemistry, 1992, Vol. 267, No. 36, pp. 26166-26171; Journal of Clinical and Experimental Medicine, 2008, Vol. 224, No. 1, pp. 51-55). See e.g., GenBank accession number: NP_{—}000236.2 (SEQ ID NO:1) for human c-MET and its α chain and β chain.

As used herein, the term “soluble c-MET” refers to a polypeptide of a partial region in the c-MET extracellular domain, which is secreted by ectodomain shedding. A cancer cell associated with the c-MET gene amplification forms a specific soluble c-MET as demonstrated in Example 1. In human, such a specific soluble c-MET consists of the partial amino acid sequence consisting of the amino acid residues at positions 1 to 922 in the amino acid sequence of SEQ ID NO:1.

Such a specific soluble c-MET is formed by the cleavage between the lysine residue (K) and the valine residue (V) in the partial region GKVI (SEQ ID NO:2) in the c-MET extracellular domain. In c-MET derived from the human, the lysine residue (K) corresponds to the amino acid residue at position 922 in the amino acid sequence of SEQ ID NO:1, and the valine residue (V) corresponds to the amino acid residue at position 923 in the amino acid sequence of SEQ ID NO:1. In the c-MET extracellular domain, the partial region GKVI (SEQ ID NO:2) to be cleaved between the lysine residue (K) and the valine residue (V) is preferably LGKVIV (SEQ ID NO:3), more preferably VLGKVIVQ (SEQ ID NO:4) and still more preferably TVLGKVIVQP (SEQ ID NO:5). The above cleavage can predominantly be caused, for example, by ADAM17 belonging to ADAM (a disintegrin and metalloprotease) family that is a one-transmembrane multi-domain protein. ADAM17 may also be called as TACE (TNF-alpha-converting enzyme).

The specific soluble c-MET formed by the above cleavage has the dipeptide unit GK at its C-terminus. The dipeptide unit possessed by such a specific soluble c-MET at its C-terminus is preferably LGK, more preferably VLGK (SEQ ID NO:6) and still more preferably TVLGK (SEQ ID NO:7).

The term “soluble (c-MET) splicing variant” refers to a polypeptide consisting of an amino acid sequence that constitutes the c-MET extracellular domain, which is formed by alternative splicing rather than ectodomain shedding. Examples of the soluble splicing variant may include variant 1 (ACF47603.1), variant 2 (ACF47604.1), variant 3 (ACF47605.1), variant 4 (ACF47606.1), variant 5 (ACF47607.1), variant 6 (ACF47608.1), variant 7 (ACF47609.1), variant 8 (ACF47610.1), variant 9 (ACF47611.1), variant 10 (ACF47612.1), variant 11 (ACF47613.1), variant 12 (ACF47614.1), variant 13 (ACF47615.1), variant 14 (ACF47616.1), and variant 15 (ACF47617.1) (see Non-patent Literature 1).

A region specific for the specific soluble c-MET refers to a region that is present in the aforementioned specific soluble c-MET but cannot be present in the soluble splicing variant. In the human, the aforementioned specific soluble c-MET consists of the partial amino acid sequence consisting of the amino acid residues at position 1 to 922 in the amino acid sequence of SEQ ID NO:1 encoding c-MET. On the other hand, any of the aforementioned soluble splicing variants has the partial region consisting of the amino acid residues at positions 1 to 861 in the amino acid sequence of SEQ ID NO:1. However, all of the soluble splicing variants cannot have the partial region consisting of the amino acid residues at positions 862 to 922 in the amino acid sequence of SEQ ID NO:1. Thus, in the human, the region specific for the specific soluble c-MET can be the partial region consisting of the amino acid residues at positions 862 to 922 in the amino acid sequence of SEQ ID NO:1.

The measurement of the specific soluble c-MET utilizing the region specific for the specific soluble c-MET can be carried out, for example, using an affinity substance for the region specific for the specific soluble c-MET. Examples of such an affinity substance may include antibodies and aptamers. The affinity substance for the region specific for the specific soluble c-MET may be, for example, an affinity substance for a peptide consisting of an amino acid sequence of QAISSTVLGK (SEQ ID NO:10) or KQAISSTVLGK (SEQ ID NO:11). The measurement of the specific soluble c-MET using such an affinity substance may also be carried out by an immunological technique. Examples of the immunological technique may include an enzyme immunoassay (EIA) (e.g., direct competitive ELISA, indirect competitive ELISA, sandwich ELISA), a radioimmunoassay (RIA), a fluorescence immunoassay (FIA), an immunochromatographic method, a luminescence immunoassay, a spin immunoassay, a western blotting method, and a latex aggregation method.

An antibody against the region specific for the specific soluble c-MET may be either a polyclonal antibody or a monoclonal antibody. The antibody may also be fragments (e.g., Fab, F(ab′)₂) of the antibody, or a recombinant antibody (e.g., scFv). The antibody may be provided in a form of being immobilized on a substrate such as a plate or a form of being immersed in a support such as a strip. The antibody can be produced by a known method per se using a peptide consisting of the region specific for the specific soluble c-MET or a partial peptide thereof as an antigen.

The polyclonal antibody can be obtained, for example, by administering the peptide consisting of the region specific for the specific soluble c-MET or the partial peptide thereof as the antigen together with a commercially available adjuvant (e.g., complete or incomplete Freund's adjuvant) subcutaneously or intraperitoneally to an animal approximately two to four times every two or three weeks, collecting whole blood about 3 to 10 days after the final immunization, and purifying antiserum. Examples of the animal administered with the antigen may include mammals such as rats, mice, rabbits, goats, cattle, guinea pigs, and hamsters.

The monoclonal antibody can be produced, for example, by a cell fusion method. For example, a mouse is administered with the peptide consisting of the region specific for the specific soluble c-MET or the partial peptide thereof together with the commercially available adjuvant subcutaneously or intraperitoneally two to four times, spleen or lymph node is taken from the mouse about 3 days after the final immunization, and leukocytes are collected. A cell fusion of this leukocyte and a myeloma cell (e.g., NS-1) is carried out to obtain a hybridoma that produces the monoclonal antibody against the factor. The cell fusion methods may include a PEG method and a voltage pulse method. The hybridoma that produces a desired monoclonal antibody can be selected by detecting an antibody specifically binding to the antigen in a culture supernatant using a well-known EIA or RIA method or the like. The hybridoma that produced the monoclonal antibody can be cultured in vitro, or in vivo such as in ascites of a mouse or a rat, preferably the mouse, and the antibody can be obtained from the culture supernatant of the hybridoma or from the ascites of the animal.

The aptamer against the region specific for the specific soluble c-MET can be produced by a technique called SELEX.

The affinity substance for the region specific for the specific soluble c-MET may be optionally provided in a form of being labeled with a labeling substance. Examples of the labeling substance may include fluorescent substances such as FITC and FAM, luminescent substances such as luminol, luciferin and lucigenin, radioisotopes such as ³H, ¹⁴C, ³²P, ³⁵S, and ¹²³I, and affinity substances such as biotin and streptoavidin.

The measurement of the specific soluble c-MET utilizing the region specific for the specific soluble c-MET may be carried out by combining an affinity substance for a c-MET fragment having the dipeptide unit VI at its N-terminus, which is formed by the cleavage between the lysine residue (K) and the valine residue (V) in the partial region GKVI (SEQ ID NO:2) in the c-MET extracellular domain, with an affinity substance for the region specific for the specific soluble c-MET. Such a combination can measure more specifically the specific soluble c-MET having the dipeptide unit GK at its C-terminus.

The measurement of the specific soluble c-MET utilizing the region specific for the specific soluble c-MET may also be carried out, for example, by treating a sample with a protease, and subsequently detecting a peptide derived from the region specific for the specific soluble c-MET. Examples of the protease may include trypsin, chymotrypsin, Lys-C, Asp-N, Glu-C, Arg-C, asparaginyl endopeptidase, arginyl endopeptidase, and V8 protease. Such a peptide is any peptide derived from the partial region consisting of the amino acid residues at positions 862 to 922 in the amino acid sequence of SEQ ID NO:1, and examples thereof may include a peptide consisting of an amino acid sequence of QAISSTVLGK (SEQ ID NO:10) or KQAISSTVLGK (SEQ ID NO:11). The detection can be carried out, for example, by a method such as HPLC or mass spectrometry.

The measurement of the specific soluble c-MET utilizing the region specific for the specific soluble c-MET may also be carried out, for example, by mass spectrometry. In the measurement by the mass spectrometry, first a sample containing the specific soluble c-MET is directly introduced into a mass spectrometer, and subsequently bombarded with gas such as argon or nitrogen to carry out collision-induced dissociation. Then, the peptide derived from the region specific for the specific soluble c-MET (e.g., a fragment of the soluble c-MET which has the dipeptide unit GK at its C-terminus) is detected in resulting fragment ions, thereby the specific soluble c-MET can be measured.

The method of the present invention is useful for examination of cancers, for example, diagnosis of cancers associated with the c-MET gene amplification. As used herein, the term “cancer” refers to a cancer that may be associated with the amplification of the c-MET gene. Examples of such a cancer may include lung cancer (e.g., non-small cell carcinoma such as squamous cell carcinoma, adenocarcinoma and large cell carcinoma, and small cell carcinoma), gastrointestinal cancer (e.g., gastric cancer, small intestine cancer, large intestine cancer, rectal cancer), pancreatic cancer, renal cancer, liver cancer, thymic cancer, spleen cancer, thyroid cancer, adrenal cancer, prostate cancer, urinary bladder cancer, ovarian cancer, uterus cancer (e.g., endometrial cancer, cervical cancer), bone cancer, skin cancer, brain tumor, sarcoma, melanoma, blastoma (e.g., neuroblastoma), adenocarcinoma, squamous cell carcinoma, solid cancer, epithelial cancer, and mesothelioma.

The sample is not particularly limited as long as the sample likely contains the specific soluble c-MET as aforementioned, and examples thereof may include liquid samples (e.g., blood, serum, urine, saliva, ascites, a tissue extract and a cell extract) and non-liquid samples (e.g., tissue samples, cell samples). Among those mentioned above, the blood, serum, urine and saliva are preferred in terms of low invasiveness. Optionally, the sample may be previously treated before the measurement. Examples of such a treatment may include centrifugation, extraction, concentration, fractionation, cell fixation, tissue fixation, tissue freezing, and tissue sectioning.

The sample is preferably the liquid sample. When the liquid sample likely contains a cell expressing full-length c-MET (in other words, membrane-bound c-MET), the method of the present invention may comprise removing such a cell. The cell can be removed, for example, by pretreatment as aforementioned (e.g., centrifugation, fractionation). The liquid sample after removing the cell can contain the specific soluble c-MET as well as different soluble c-MET and the soluble splicing variant, but does not contain the full-length c-MET (i.e., membrane-bound c-MET having the region specific for the specific soluble c-MET as aforementioned). Thus, by utilizing the region specific for the specific soluble c-MET, it is possible to specifically measure the specific soluble c-MET.

Examples of a subject from whom the sample is derived may include a mammal that is suffered from the cancer or a mammal that is suspected of being suffered from the cancer. Examples of the mammal may include primates, pet animals, and working animals. Specifically, examples of the mammal may include humans, chimpanzees, dogs, cats, cattle, sheep, horses, pigs, rabbits, mice, and rats, and the mammal is preferably the human.

A subject having the cancer associated with the c-MET gene amplification is thought to abundantly express the substrate protein c-MET which is to be cleaved by a specific protease (e.g., ADAM17) due to the c-MET gene amplification. In such a subject, it is thought that correlating with abundantly expressed c-MET, a concentration of the specific soluble c-MET formed by cleavage of c-MET is also increased. This time, it has been found that the specific soluble c-MET is secreted by the cleavage between the lysine residue (K) and the valine residue (V) in the partial region GKVI (SEQ ID NO:2) in the extracellular domain. In order to diagnose the cancer associated with the c-MET gene amplification with a high degree of accuracy, it is necessary to distinctively measure the specific soluble c-MET which can be secreted from the cancer cell associated with the c-MET gene amplification from different soluble c-MET and the soluble splicing variant which can be secreted from the cell not associated with the c-MET gene amplification. It is thought that the aforementioned concentration of the specific soluble c-MET can be utilized as an indicator for the presence or absence and an amount of the c-MET gene amplification cell and further an indicator for determining with a high degree of accuracy whether being suffered from the cancer associated with c-MET gene amplification or not.

The subject from whom the sample is derived may be a patient with cancer for whom the treatment with a specific anticancer agent is considered. Examples of the specific anticancer agent may include a c-MET inhibitor and anticancer agents other than the c-MET inhibitor. Examples of the c-MET inhibitor may include PHA-665752, SU11274, XL-880, XL-184, ARQ 197, AMG208, AMG458, CE-355621, and MP470. Examples of the anticancer agents other than the c-MET inhibitor may include an EGFR inhibitor, an ALK inhibitor, a PDGFR inhibitor, and a c-KIT inhibitor. Example of the EGFR inhibitor may include gefitinib, erlotinib, cetuximab, lapatinib, ZD6474, CL-387785, HKI-272, XL647, PD153035, CI-1033, AEE788, BIBW-2992, EKB-569, and PF-299804. Examples of the ALK inhibitor may include WHI-P154, TAE684, and PF-2341066. Examples of the PDGFR inhibitor may include Gleevec, Desatinib, Valatinib, and Pazopanib. Examples of the c-KIT inhibitor may include Imatinib, Sunitinib, Valatinib, Desatinib, Masitinib, Motesanib, and Pazopanib. The method of the present invention is useful for selecting patients having cancers associated with the c-MET gene amplification that are sensitive to the c-MET inhibitor or resistant to the anticancer agent other than c-MET and patients having cancers not associated with the c-MET gene amplification that are resistant to the c-MET inhibitor or sensitive to the anticancer agent other than c-MET. For example, see International Publication WO2011/125458 that discloses that the cancer sensitive to the c-MET inhibitor, which can be associated with the c-MET gene amplification and is sensitive to the c-MET inhibitor can be specifically determined by measuring the concentration of the c-MET extracellular domain fragment (specific soluble c-MET).

The subject from whom the sample is derived may be a patient with cancer for whom a therapeutic effect of the specific anticancer agent is monitored. The patient with cancer may also be a patient who is required to evaluate whether a treatment with the specific anticancer agent should be switched to a treatment with another anticancer agent when a reduction of the therapeutic effect of the specific anticancer agent is observed. The anticancer agent can be the same as those described above.

In a specific embodiment, the method of the present invention may comprise:

(1) treating a sample with a protease; and
(2) measuring a fragment of the soluble c-MET which has the dipeptide unit GK at its C-terminus.

In (1), the sample is preferably the aforementioned liquid sample. When the liquid sample likely contains the cell expressing the full-length c-MET (in other words, membrane-bound c-MET), the method of the present invention may comprise removing such a cell as aforementioned.

When the sample is treated with the protease as aforementioned, the fragment of the soluble c-MET having the dipeptide unit GK at its C-terminus that is derived from the specific soluble c-MET which can be contained in the sample is produced. On the other hand, even if the different soluble c-MET and the soluble splicing variant are treated with the protease, no fragment of the soluble c-MET having the dipeptide unit GK at its C-terminus is produced. Thus, by measuring the fragment of the specific soluble c-MET having the dipeptide unit GK at its C-terminus, it is possible to evaluate the presence or absence and the amount of the specific soluble c-MET in the sample, and further it is also possible to qualitatively or quantitatively detect the c-MET amplification cell in the subject from whom the sample is derived.

The present invention also provides an affinity substance for the region specific for the soluble c-MET formed by the cleavage between the lysine residue (K) and the valine residue (V) in the partial region GKVI (SEQ ID NO:2) in the c-MET extracellular domain. The region specific for the soluble c-MET formed by the cleavage between the lysine residue (K) and the valine residue (V) in the partial region GKVI (SEQ ID NO:2) in the c-MET extracellular domain is as aforementioned, and preferably a peptide region consisting of an amino acid sequence of SEQ ID NO:10 or SEQ ID NO:11. The affinity substance is as aforementioned, preferably an antibody and more preferably a monoclonal antibody. The affinity substance may optionally be provided in the form of being labeled with the labeling substance as described above.

The present invention further provides a diagnostic agent for cancers comprising the affinity substance as described above. The cancer and the subject to be diagnosed are as described above.

The reagent of the present invention may be provided in a form of a kit comprising an additional component in addition to the affinity substance as described above. In this case, respective components contained in the kit may be provided in a mutually separated form, e.g., a form of being contained in a different vessel (e.g., tube). For example, when the affinity substance is not labeled with the labeling substance, such a kit may further comprise the labeling substance. Such a kit may also comprise the affinity substance for the fragment of the c-MET having the dipeptide unit VI at its N-terminus, which is formed by the above cleavage. Such a kit may further comprise the specific soluble c-MET as described above or a partial peptide thereof as a positive control.

When the reagent of the present invention is provided in the form of the kit, the kit may comprise an additional component depending on a type of the affinity substance as described above. For example, when the affinity substance is an antibody, the kit may further comprise a secondary antibody (e.g., anti-IgG antibody) and a reagent for detecting the secondary antibody.

When the reagent of the present invention is provided in the form of the kit, the kit may further comprise an instrument capable of collecting the sample such as the liquid sample. The instrument capable of collecting the sample is not particularly limited as long as the sample can be obtained from the subject, and examples of the instrument may include a blood collecting instrument such as a syringe.

### EXAMPLES

The present invention will be described in more detail with reference to following Examples, but the present invention is not limited thereto.

### Example 1

### Secretion of Soluble c-MET by ADAM17

A negative control si-RNA (si-N.C.) for the c-MET gene amplification lung cancer cell line NCI-H1993 and si-RNA (si-AD10 or si-AD17) targeting an ADAM10 gene or an ADAM17 gene were transfected. At a time point of 24 hours after the transfection, a medium was changed to a serum-free medium, and a culture supernatant after incubating the serum-free medium for 24 hours was collected. The culture supernatant filtrated through a filter was concentrated through an ultrafiltration membrane, subsequently developed on SDS-PAGE, and transferred onto a PVDF membrane. An effect of ADAM10 and ADAM17 knockdown on secretion of soluble c-MET was confirmed by a western blotting method using an antibody that recognized an extracellular domain (MET-α chain).

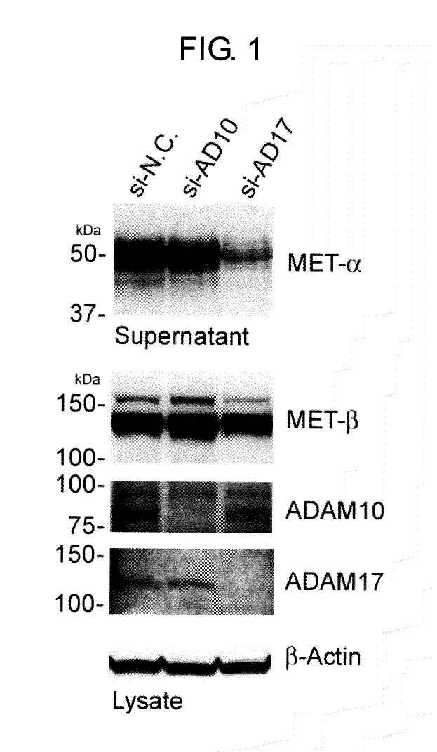

As a result, the secretion of c-MET was remarkably inhibited by the knockdown of ADAM17 whereas it was not inhibited by the knockdown of ADAM10 (FIG. 1). Thus, it has been shown that the specific soluble c-MET specific for a cancer cell, which is produced by cleavage of c-MET, is secreted mainly through a cleavage action of ADAM17.

### Example 2

### Concentration of Soluble c-MET by Immunoprecipitation

The c-MET gene amplification lung cancer cell line NCI-H1993 at a sub-confluent state was incubated in the serum-free medium for 7 days, and subsequently a culture supernatant was collected. The culture supernatant filtrated through the filter was concentrated through the ultrafiltration membrane, and subsequently immunoprecipitated with an antibody that recognized an extracellular region of c-MET. The resulting immunoprecipitation complex was developed on non-reduced SDS-PAGE, and stained with a biosafety CBB staining solution.

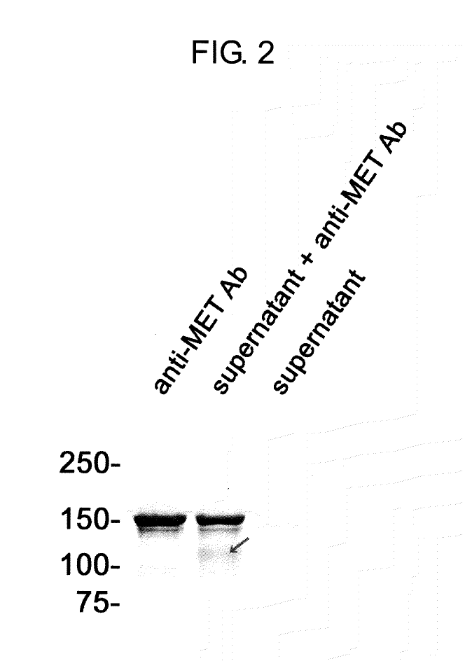

As a result, a band that was thought to correspond to the specific soluble c-MET (a fragment of a c-MET extracellular domain) produced by the cleavage of c-MET expressing in cancer cells, which was different from a band for the antibody of 150 kDa, was detected in a sample in which the culture supernatant had been mixed with the antibody (FIG. 2).

### Example 3

### Identification of C-Terminus Candidate of Soluble c-MET by LC-Tandem Mass Spectrometry

A stained portion exhibiting a mobility corresponding to the soluble c-MET was cut out using a cutter, and digested with trypsin or chymotrypsin in the gel. The resulting digested product was analyzed by an LC-tandem mass apparatus (Thermo LTQ XL), and SEQUEST analysis was carried out by utilizing data set of a polypeptide consisting of a virtual sequence of the soluble c-MET as a reference sequence.

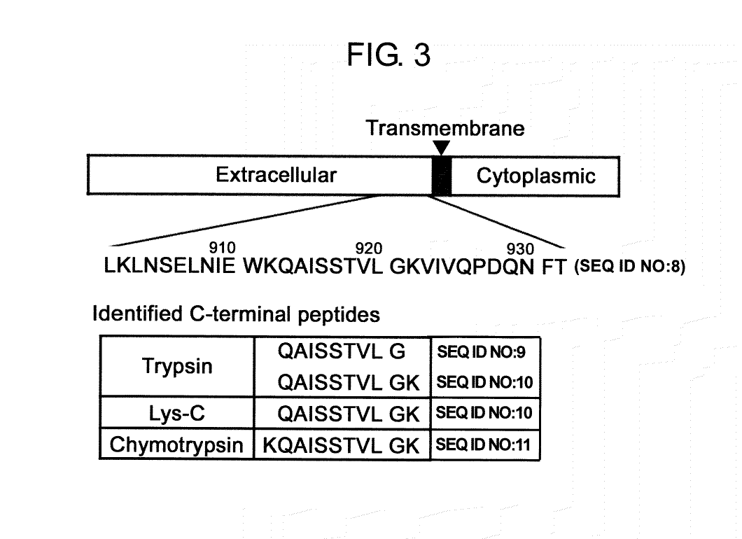

As a result, a peptide fragment having 921G and 922K at its C-terminus was detected in the product digested with trypsin, and a peptide fragment having 922K at its C-terminus was detected in the product digested with Lys-C (FIG. 3). A peptide fragment having 922K at its C-terminus was detected in the product digested with chymotrypsin. From sequence recognition profiles of trypsin, Lys-C and chymotrypsin, 921G was thought to be highly likely due to error digestion with trypsin or error recognition by the mass spectrometer. Thus, it has been shown that the amino acid residue at the C-terminus of the specific soluble c-MET secreted by the c-MET gene amplification cell is 922K as well as such a specific soluble c-MET is formed by the cleavage between the lysine residue (K) and the valine residue (V) in the partial region GKVI (SEQ ID NO:2) in the extracellular domain.

### Example 4

### Tandem Mass Spectrum of C-Terminus Candidate Peptide

A tandem mass spectrum derived from a C-terminal peptide contained in the product digested with trypsin or the product digested with chymotrypsin was identified based on information for m/z values of predicted precursor ions and product ions from all tandem mass spectrum data detected from soluble c-MET samples. The product ion having the predicted m/z value was imputed to each spectrum, and it was proven that the digested products contained a fragment digested with trypsin: QAISSTVLGK (SEQ ID NO:10) and a fragment digested with chymotrypsin: KQAISSTVLGK (SEQ ID NO:11).

Therefore, it has been shown that the soluble c-MET is formed by the cleavage between the lysine residue (K) and the valine residue (V) in the partial region GKVI (SEQ ID NO:2) in the extracellular domain in the c-MET gene amplification cell.

### INDUSTRIAL APPLICABILITY

The method of present invention is useful for examining the cancers, and the like.

1. A method for detecting a c-MET gene amplification cell, the method comprising:
measuring an amount of a soluble c-MET in a sample by utilizing a region specific for the soluble c-MET,
wherein the soluble c-MET is formed by cleavage between a lysine residue (K) and a valine residue (V) in a partial region GKVI (SEQ ID NO:2) in a c-MET extracellular domain.

2. The method according to claim 1,
wherein the method is a method for examining a cancer.

3. The method according to claim 1,
wherein the sample is derived from a human.

4. The method according to claim 1,
wherein the sample is a liquid sample.

5. The method according to claim 1,
wherein the c-MET gene amplification cell is a lung cancer cell.

6. The method according to claim 1, comprising:
(1) treating the sample with a protease; and
(2) measuring the amount of a soluble c-MET fragment having a dipeptide unit GK at its C-terminus.

7. The method according to claim 1,
wherein the measuring the soluble c-MET is carried out by detecting a soluble c-MET fragment having a dipeptide unit GK at its C-terminus with mass spectrometry.

8. The method according to claim 6,
wherein the soluble c-MET fragment is a peptide selected from the group consisting of an amino acid sequence of QAISSTVLGK (SEQ ID NO:10) and KQAISSTVLGK (SEQ ID NO:11).

9. An affinity substance for a region specific for a soluble c-MET formed by cleavage between a lysine residue (K) and a valine residue (V) in a partial region GKVI (SEQ ID NO:2) in a c-MET extracellular domain.

10. The affinity substance according to claim 9,
wherein the affinity substance is an antibody.

11. A diagnostic reagent for cancers, comprising an affinity substance for a region specific for a soluble c-MET formed by cleavage between a lysine residue (K) and a valine residue (V) in a partial region GKVI (SEQ ID NO:2) in a c-MET extracellular domain.


SEQUENCE LISTING (11 sequences)

SEQ ID NO: 1 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 2 - protein, 4 aa, Artificial Sequence
  Partial amino acid sequence from human c-MET
GKVI

SEQ ID NO: 3 - protein, 6 aa, Artificial Sequence
  Partial amino acid sequence from human c-MET
LGKVIV

SEQ ID NO: 4 - protein, 8 aa, Artificial Sequence
  Partial amino acid sequence from human c-MET
VLGKVIVQ

SEQ ID NO: 5 - protein, 10 aa, Artificial Sequence
  Partial amino acid sequence from human c-MET
TVLGKVIVQP

SEQ ID NO: 6 - protein, 4 aa, Artificial Sequence
  Partial amino acid sequence from human c-MET
VLGK

SEQ ID NO: 7 - protein, 5 aa, Artificial Sequence
  Partial amino acid sequence from human c-MET
TVLGK

SEQ ID NO: 8 - protein, 32 aa, Artificial Sequence
  Partial amino acid sequence from human c-MET
LKLNSELNIEWKQAISSTVLGKVIVQPDQNFT

SEQ ID NO: 9 - protein, 9 aa, Artificial Sequence
  Partial amino acid sequence from human c-MET
QAISSTVLG

SEQ ID NO: 10 - protein, 10 aa, Artificial Sequence
  Partial amino acid sequence from human c-MET
QAISSTVLGK

SEQ ID NO: 11 - protein, 11 aa, Artificial Sequence
  Partial amino acid sequence from human c-MET
KQAISSTVLGK